(19) United States
(12) Patent Application Publication
(10) Pub. No.: US 2010/0179202 A1
(43) Pub. Date: Jul. 15, 2010

(54) ORALLY DISINTEGRATING COMPOSITION OF ZOLMITRIPTAN

(75) Inventors: Meenakshi Patnaik, Mumbai (IN); Shailesh Singh, Mumbai (IN); Kumar Nitin Swarnakar, Mumbai (IN); Eswaran Iyer, Mumbai (IN)

Correspondence Address: BIO INTELLECTUAL PROPERTY SERVICES (BIO IPS) LLC, 8509 KERNON CT., LORTON, VA, 22079, US

(73) Assignee: AJANTA PHARMA LIMITED.

(21) Appl. No.: 12/687,175

(22) Filed: Jan. 14, 2010

Related U.S. Application Data
(60) Provisional application No. 61/193,973, filed on Jan. 14, 2009.

(51) Int. Cl. A61K 31/422; A61P 25/06

(52) U.S. Cl. 514/376

(57) ABSTRACT

A composition containing Zolmitriptan in the form of oral disintegrating tablet for the acute treatment of migraines is disclosed. The said composition comprises (a) Zolmitriptan, a salt or solvate or polymorph thereof as active ingredient, (b) mannitol, and (c) calcium silicate, and (d) optionally, one or more polysaccharides along with one or more pharmaceutically acceptable excipients.

### CROSS REFERENCE TO RELATED APPLICATIONS:

This application claims the benefit of U.S. Provisional Application No. 61/193,973, filed Jan. 14, 2009. The entire disclosure of this prior application is hereby incorporated by reference.

### FEDERALLY SPONSORED RESEARCH

Not applicable

### SEQUENCE LISTING OR PROGRAM

Not Applicable.

### FIELD OF THE INVENTION

The present invention discloses a composition containing Zolmitriptan in the form of an oral disintegrating tablet for the acute treatment of migraines.

### BACKGROUND OF THE INVENTION

The information provided below is not admitted to be prior art to the present invention, but is provided solely to assist the understanding of the reader.

Migraine headaches are a major public health problem. The impact of these headaches on patients and their families is tremendous, with many patients reporting frequent and significant disability.

The U.S. Pat. No. 5,466,699 discloses Zolmitriptan as a serotonin (selective 5-HT1B/1D) receptor agonist of the 1B and 1D subtype, used in the acute treatment of migraine attacks with or without aura and cluster headaches. During migraine occurrences excessive cerebrovascular dilation and neutrogena inflammatory processes are considered to contribute to the level and extent of pain. The 5-HT1B/1D-receptors mediate cerebrovascular vasoconstriction and inhibit neurogenic inflammation. 5-HT1B/1D receptor agonists are beneficial in the treatment (including prophylaxis) of disease conditions wherein vasoconstriction and neurogenic inflammation in the cerebrovascular bed is indicated, for example migraine, cluster headache and headache associated with vascular disorders, hereinafter referred to collectively as migraine. Zolmitriptan has been developed for the acute treatment of migraine in the form of a 2.5 mg and 5 mg tablet intended to be taken up to a maximum of 15 mg per day.

Chemically, Zolmitriptan is (4S)-4-{[3-(2-dimethylaminoethyl)-1H-indol-5-yl]methyl}-1,3-oxazolidin-2-one (CAS No. 139264-17-8). The chemical structure of Zolmitriptan can be represented as Formula I

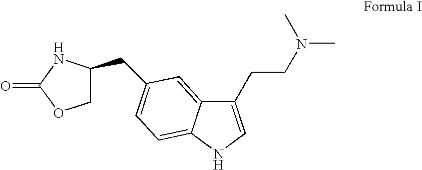

Zolmitriptan is a synthetic tryptamine derivative and appears as a white powder that is readily soluble in water.

Zolmitriptan was developed by Burroughs Wellcome Co. as a new chemical compound useful for the prophylaxis and treatment of migraine and is currently marketed by Astra Zenica as ZOMIG-ZMT™ Orally Disintegrating Tablets. ZOMIG-ZMT™ Orally Disintegrating Tablets are available as 2.5 mg white uncoated tablets for oral administration. The orally disintegrating tablets contain mannitol USP, microcrystalline cellulose NF, crospovidone NF, aspartame NF, sodium bicarbonate USP, citric acid unhydrous USP, colloidal silicon dioxide NF, magnesium stearate NF, and orange flavor SN 027512.

Orally disintegrating dosage forms are becoming an increasingly important issue in the area of better patient compliance comparative to the conventional solid dosage forms such as capsules and tablets for oral administration, which are the most commonly used. In particular pediatric and geriatric patients often experience difficulties in swallowing solid dosage forms. Also, conventional solid dosage forms are not suitable for bedridden or busy and travelling patients, who may not have access to water. Thus orally dispersible tablets represent an alternative for those patients and provide for a better patient compliance with recommended pharmaceutical therapies.

Orally disintegrating tablets containing olanzapine are known and sold under the trade name Zyprexa® Zydis™. They are prepared by a freeze-drying technique, as is for example described in EP435684A1. Freeze drying on a large scale has not been found to be very effective. Moreover, it is a time consuming technique. The Zydis tablets prepared by this technique are so fragile that the formation of the matrix material has to take place in a specific container. Tablets manufactured by this technology require a special type of packaging and careful handling during dispensing and administration to the patients, since they are prone to breakage.

Orally disintegrating tablets were described in the various patent applications such as WO 03/103629, EP1323417A1, WO 03/086361, EP1120109A2, EP1260215A1 and WO 06/074951.

Zolmitriptan being a drug of choice in acute treatment of migraine attack, it is expected to be formulated in a convenient dosage form capable of delivering the drug to the patient's system immediately after administration for immediate relief from the painful attack of the migraine.

The present invention relates to an orally disintegrating composition, in particular in the form of tablets, which contains Zolmitriptan, and a process for manufacture of such a composition.

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, and from the claims.

For clarity of disclosure, and not by way of limitation, the detailed description of the invention is divided into the subsections that follow.

This invention is directed to an orally disintegrating pharmaceutical composition comprising (a) Zolmitriptan, a salt or solvate or polymorph thereof as active ingredient, (b) mannitol, and (c) calcium silicate and (d) optionally, one or more polysaccharides along with one or more pharmaceutically acceptable excipients.

The term “orally disintegrating” means that the composition disintegrates or disperses within 90 seconds as measured by the in vitro disintegration test.

The composition according to the invention preferably disintegrates in less than 60 seconds, and more preferably in less than 30 seconds.

Preferably, the composition takes the form of a tablet. Such a tablet has preferably a mass of less than 250 mg.

It is also preferred that a single dosage form of the composition according to the invention, such as a tablet or capsule, comprises 1 to 10 mg of Zolmitriptan or salt or solvate thereof, calculated as Zolmitriptan.

The calcium silicate used in the composition can be in crystalline or amorphous form or a mixture thereof. The particle size of the calcium silicate is preferably in the range from 1 to 500 μm. The average particle size is preferably 1 to 100 μm.

The composition comprises 5 to 50% by weight of calcium silicate and preferably 10 to 30% by weight of calcium silicate.

The composition optionally contains polysaccharide such as starch, pregelatinized starch, cellulose and mixtures thereof.

The composition usually comprises at least one further excipient, other than mannitol and calcium silicate, selected from the group of disintegrants, binders, fillers, flavouring agents, sweetening agents, glidants, colouring agents and lubricants.

The disintegrant is preferably selected from the group consisting of crospovidone, croscarmellose sodium, sodium starch glycolate low-substituted hydroxypropyl cellulose or pregelatinized starch. Preferably the disintegrant is crospovidone or low substituted hydroxypropyl cellulose or a combination of both.

The binders used in the composition of the invention are preferably selected from gelatin, pregelatinized starch, starch DC, acacia, tragacanth, guar gum, hydroxypropyl cellulose, low-substituted hydroxypropyl cellulose, hydroxypropylmethyl cellulose, methylcellulose, glucose, sucrose, sorbitol, polyvinyl pyrrolidone and the like. The preferred binder is low-substituted hydroxypropyl cellulose.

Suitable fillers are preferably selected from at least one of starch derivatives, such as corn starch, potato starch or rice starch; polysaccharides such as dextrins, maltodextrins, dextrates, microcrystalline cellulose, powdered cellulose, mixtures of microcrystalline cellulose and guar gum, coprocessed blends of microcrystalline cellulose; and polyhydric alcohols, such as xylitol and sorbitol. The preferred filler is corn (maize) starch and microcrystalline cellulose.

Suitable sweeteners include sugars, such as sucrose, lactose and glucose; cyclamate and salts thereof; saccharin and salts thereof; and aspartame. The preferred sweetener is aspartame.

The composition may, additionally, include flavouring agents which can be natural or synthetic flavours such as strawberry flavour, wild cherry flavour, green apple flavour, spearmint flavour, and peppermint flavour as well as glidants such as aerosil (fumed silicon dioxide), starch, talc, and magnesium stearate. The preferred glidants is aerosil. Optionally, the composition may include one ore more lubricants which are magnesium stearate, stearic acid, sodium stearyl fumarate, magnesium lauryl sulphate, polyethylene glycol, and glyceryl behenate.

The preferred lubricant is magnesium stearate.

In one embodiment, the active ingredient, mannitol and the calcium silicate are blended together and the blend is then mixed with other ingredients and the resulting mixture then finally subjected to direct compression to form a tablet dosage form.

It is a very advantageous aspect of the invention that the compositions of the thermo-labile and moisture sensitive compounds such as Zolmitriptan are prepared without subjecting the active ingredient to the moisture by a wet granulation step and the elevated temperatures used in the drying procedure usually concluding the wet granulation. Moreover, as the active ingredient does not need to be subjected to wet granulation their polymorphic form remains unchanged which is a further significant benefit of the composition according to the invention.

Moreover, the composition according to the invention has sufficient strength and only a low friability of preferably less than 1% w/w allowing for example tablets to be packed into regular containers, such as bottles, blisters, strip packs or sachets, and to be stored in bulk in drums.

### EXAMPLE 1

| Sr. No. | Ingredient | mg/tablet |
|---|---|---|
| 1 | Zolmitriptan | 5.00 |
| 2 | Mannitol | 137.15 |
| 3 | Calcium Silicate | 31.25 |
| 4 | Crospovidone | 20 |
| 5 | Aspartame | 3.00 |
| 6 | Silicon Dioxide (Aerosil) | 1.60 |
| 7 | Magnesium Stearate | 2.00 |
| | Total weight | 200 |

The ingredients 1 to 3 were weighed, sifted, and mixed in a blender. Ingredients 4 to 6 are weighed, sifted and mixed into the above blend. The resulting blend is then lubricated with magnesium stearate and silicon dioxide in a blender. The lubricated blend is then compressed in to tablets using a Cadmach Compression Machine.

### EXAMPLE 2

| Sr. No. | Ingredient | mg/tablet |
|---|---|---|
| 1 | Zolmitriptan | 5.00 |
| 2 | Mannitol | 86.25 |
| 3 | Calcium Silicate | 28.75 |
| 4 | Maize starch | 36.4 |
| 5 | Crospovidone | 15.00 |
| 6 | Microcrystalline Cellulose | 22.00 |
| 7 | Aspartame | 3.00 |
| 8 | Silicon Dioxide (Aerosil) | 1.60 |
| 9 | Magnesium Stearate | 2.00 |
| | Total weight | 200 |

The ingredients 1 to 7 were weighed, sifted, and mixed in a V-blender. The initial blend is lubricated with Silicon Dioxide and Magnesium Stearate in an octagonal blender. The lubricated blend is then compressed in to tablets using a Cadmach Compression Machine.

### EXAMPLE 3

| Sr. No. | Ingredient | mg/tablet |
|---|---|---|
| 1 | Zolmitriptan | 2.5 |
| 2 | Mannitol | 68.575 |
| 3 | Calcium Silicate | 15.625 |
| 4 | Crospovidone | 10 |
| 5 | Aspartame | 1.5 |
| 6 | Silicon Dioxide (Aerosil) | 0.8 |
| 7 | Magnesium Stearate | 1 |
| | Total weight | 100 |

The ingredients 1 to 3 were weighed, sifted, and mixed in a blender. Ingredients 4 to 6 are weighed, sifted and mixed into the above blend. The resulting blend is then lubricated with magnesium stearate and silicon dioxide in a blender. The lubricated blend is then compressed in to tablets using a Cadmach Compression Machine.

### EXAMPLE 4

| Sr. No. | Ingredient | mg/tablet |
|---|---|---|
| 1 | Zolmitriptan | 2.5 |
| 2 | Mannitol | 43.125 |
| 3 | Calcium Silicate | 14.375 |
| 4 | Maize starch | 18.2 |
| 5 | Crospovidone | 7.5 |
| 6 | Microcrystalline Cellulose | 11 |
| 7 | Aspartame | 1.5 |
| 8 | Silicon Dioxide (Aerosil) | 0.8 |
| 9 | Magnesium Stearate | 1 |
| | Total weight | 100 |

The ingredients 1 to 7 were weighed, sifted, and mixed in a V-blender. The initial blend is lubricated with Silicon Dioxide and Magnesium Stearate in an octagonal blender. The lubricated blend is then compressed in to tablets using a Cadmach Compression Machine.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. An orally disintegrating pharmaceutical composition which comprises (a) Zolmitriptan, a salt or solvate or polymorph thereof as the active ingredient, (b) mannitol, (c) calcium silicate, and (d) optionally, one or more polysaccharides and/or one or more pharmaceutically acceptable excipients.

2. The composition according to claim 1 wherein mannitol is selected from pearlitol 160 and pearlitol 200 SD.

3. The composition according to claim 1 which comprises 30 to 90% by weight of mannitol.

4. The composition according to claim 1 which comprises 5 to 40% by weight of calcium silicate.

5. The composition according to claim 1 which comprises 5 to 40% by weight of one or more polysaccharides

6. The composition according to claim 1 wherein the one or more polysaccharide is selected from starch, pregelatinized starch, cellulose and mixtures thereof.

7. A process for preparing an orally disintegrating composition comprising (a) Zolmitriptan, a salt or solvate or polymorph thereof as active ingredient, (b) mannitol, (c) calcium silicate and (d) optionally one or more polysaccharides and/or one or more pharmaceutically acceptable excipients by direct compression.

8. The process according to claim 7, wherein direct compression method comprises
(a) mixing of weighed and sifted active ingredient with mannitol, calcium silicate and optionally other pharmaceutically acceptable excipients,
(b) lubricating the blend of (a), and
(c) compressing the lubricated blend in to tablets.

9. The composition according to claim 1, which disintegrates in less than 60 seconds in the oral cavity.

10. A method of treating migraine in a patient in need thereof comprising administering the composition according to claim 1.
